(19) United States
(12) Patent Application Publication
(10) Pub. No.: US 2023/0056301 A1
(43) Pub. Date: Feb. 23, 2023

(54) COMPOSITIONS AND METHODS FOR DELIVERING THERAPEUTIC ANTIBODIES USING PLATELET-DERIVED MICROPARTICLES

(71) Applicant: North Carolina State University, Raleigh, NC (US)

(72) Inventors: Ke Cheng, Raleigh, NC (US); Zhenhua Li, Raleigh, NC (US)

(21) Appl. No.: 17/785,754

(22) PCT Filed: Dec. 15, 2020
(86) PCT No.: PCT/US2020/065089

§ 371 Date: Jun. 15, 2022

Related U.S. Application Data
(60) Provisional application No. 62/948,637, filed on Dec. 16, 2019.

(51) Int. Cl. C07K 16/24; A61K 47/69; A61K 47/10; A61P 29/00

(52) U.S. Cl. CPC C07K 16/245; A61K 47/6901; A61K 47/10; A61P 29/00; A61K 2039/505

(57) ABSTRACT

The present disclosure provides compositions and methods relating to the use of platelet microparticles to deliver therapeutic antibodies. In particular, the present disclosure provides novel compositions and methods for treating cardiac injury using anti-IL-1β platelet microparticles (IL1-PMs) to promote cardiac detoxification and repair after cardiac injury (e.g., myocardial infarction).

### CROSS REFERENCE TO RELATED APPLICATIONS

This application claims priority to and the benefit of U.S. Provisional Patent Application No. 62/948,637 filed Dec. 16, 2019, which is incorporated herein by reference in its entirety for all purposes.

### FIELD

The present disclosure provides compositions and methods relating to the use of platelet microparticles to deliver therapeutic antibodies. In particular, the present disclosure provides novel compositions and methods for treating cardiac injury using anti-IL-1β platelet microparticles (IL1-PMs) to promote cardiac detoxification and repair after cardiac injury (e.g., myocardial infarction).

### BACKGROUND

Acute myocardial infarctions (AMI), principally caused by the occlusion of a coronary artery, are a major cause of death and disability worldwide. Myocardial infarctions (MI) induce the reduction of blood flow to heart muscles and result in myocardial necrosis. The myocardial necrosis triggers an inflammatory response that contributes to adverse left ventricular (LV) remodeling and heart failure. Therefore, inhibition of the inflammatory response might serve as a potent strategy for the prevention of adverse cardiac remodeling and eventual heart failure.

During the course of an immune response, inflammasomes closely regulate the activation of caspase-1, an enzyme that is primarily responsible for processing and activating powerful pro-inflammatory cytokines, such as IL-1β and IL-18. Among these pro-inflammatory cytokines, IL-1β plays a central role in the sterile inflammatory response resulting from MI by promoting the synthesis of other proinflammatory cytokines, activating profibrotic pathways, and promoting cardiomyocyte apoptosis. The interest in IL-1β as a therapeutic target has led to the development of several IL-1β blockers that interrupt IL-1 signaling. Such blockers include IL-1 receptor antagonists, anti-IL-1β-neutralizing antibodies, and decoy receptors. However, none of IL-1β blockers have been approved for clinical application in MI patients at the present time. That includes Canakinumab (Ilaris), an IL-1β antibody that entered the clinical stage (Anti-Inflammatory Thrombosis Outcomes Study (CANTOS)) in 2017. One major reason for the lack of successful clinical candidates is the poor safety profile of IL-1β blockers. For example, he application of IL-1β blockers may increase the risk of fatal infections due to their lack of targeting capacity, which blunts the body's local and systemic inflammatory response to infection. Therefore, there is a need for therapeutic intervention that overcomes these limitations.

### SUMMARY

Embodiments of the present disclosure include a platelet-derived microparticle comprising a linker moiety and at least one therapeutic antibody.

In some embodiments, the linker moiety is functionally coupled to the surface of the microparticle. In some embodiments, the linker moiety is functionally coupled to the surface of the microparticle via a lipophilic headgroup. In some embodiments, the linker moiety comprises 1,2-Distearoyl-sn-glycero-3-phosphorylethanolamine (DSPE) and a PEG polymer.

In some embodiments, the PEG polymer is at least 5 kDa. In some embodiments, the linker moiety comprises an NHS-terminated DSPE-PEG polymer. In some embodiments, the at least one therapeutic antibody is covalently bound to the linker moiety.

In some embodiments, the at least one therapeutic antibody neutralizes at least one aspect of an immune response. In some embodiments, the at least one therapeutic antibody is a monoclonal antibody. In some embodiments, the at least one therapeutic antibody binds to IL-1β. In some embodiments, the at least one antibody is Gevokizumab, Canakinumab, and any derivatives, variants, or combinations thereof.

In some embodiments, the microparticle includes at least a second therapeutic antibody. In some embodiments, the second therapeutic antibody targets at least one of IL-1α, IL-16, IL-18, and TNF-α.

In some embodiments, the microparticles are derived from inactivated platelets.

In some embodiments, the microparticle includes at least one therapeutic agent (e.g., agent that treats an MI).

Embodiments of the present disclosure also include a composition comprising a plurality of microparticles described above, and at least one pharmaceutically acceptable carrier or excipient.

In some embodiments, the composition further includes a physiologically suitable buffer. In some embodiments, the composition further includes at least one therapeutic agent (e.g., agent that treats an MI).

Embodiments of the present disclosure also include a method for treating a subject that has suffered a cardiac event. In accordance with these embodiments, the method includes administering the composition described above to a subject in need thereof. In some embodiments, the composition is administered intravenously, subcutaneously, intramuscularly, or by surgical intervention.

### BRIEF DESCRIPTION OF THE DRAWINGS

FIG. 1: Schematic diagram. Schematic illustrating the role of Gevokizumab-armed platelet microparticles as cardiac detoxification and repair agents.

FIGS. 2A-2C: Biodistribution of IL1-PMs in mice with acute MI. (A) In vivo fluorescent imaging of myocardial infarction (MI) mice or sham mice at various time intervals after i.v. injection of IL1-PM@Cy5.5 or antibody@Cy5.5. (B) Ex vivo fluorescent imaging of the major organs excised from the treated animals. (C) Quantitative analysis of fluorescent intensity in the organs. MI, myocardial infarction; Antibody, Gevokizumab; IL1-PM, Gevokizumab-armed platelet microparticles. **p<0.01 indicates that the IL1-PM@Cy5.5 treated MI group is significantly different from the other groups.

FIGS. 3A-3C: Effects of IL1-PM treatment on inflammatory cytokines. (A) Cytokine array analysis of the systemic inflammatory cytokine level changes after 72 h of treatment. (B) Quantitative summary of cytokine array analysis in A. (C) Quantitative summary of the concentrations of IL-1β in the heart as detected by ELISA (n=5). P, platelets; Antibody, Gevokizumab; IL1-PM, Gevokizumab-armed platelet microparticles. **p<0.01, ***p<0.001.

FIGS. 4A-4E: Anti-inflammatory ability of IL1-PMs in heart tissue. Western blot results for CD45 (A) and cleaved caspase-1 (B) presence in the plasma 72 h after surgery (n=3); (C) Histogram summarizing caspase-1 (YVAD-AMC cleavage) activity normalized to the PBS group (n=5). (D) Quantification of the number of ASC-positive inflammasomes; (E) Representative image of the formation of ASC-containing inflammasomes 72 h after MI. HPF means high powered field. ns, no significance. ASC, apoptosis-associated speck-like protein containing a caspase recruitment domain; P, platelets; Antibody, Gevokizumab; IL1-PM, Gevokizumab-armed platelet microparticles.

FIGS. 5A-5D: IL1-PM treatment reduces cardiac apoptosis. (A) Expression of apoptosis-associated protein (caspase-3) analyzed by western blot 72 h after treatment; (B) Caspase-3 activity was evaluated using a fluorometric assay kit (n=5); (C) TUNEL staining for cardiomyocyte apoptosis in the infarcted heart 3 days after MI. Scale bar: 20 μm. (D) Quantification of cardiomyocyte apoptosis. P, platelets; Antibody, Gevokizumab; IL1-PM, Gevokizumab-armed platelet microparticles. ***p<0.001.

FIGS. 6A-6G: IL1-PM treatment attenuates cardiac remodeling. (A) Representative Masson's trichrome staining of myocardial sections 70 days after treatment. Quantitative analyses of (B) viable myocardium and (C) scar size from the Masson's trichrome images. IL1-PM groups vs other three groups. (D) Left ventricular end-diastolic volume (LVEDV) and (E) left ventricular end-systolic volume (LVESV) measured by echocardiography 4 hours, 28 days, and 70 days after treatment (n=5). (F) LVEFs and (G) LVFSs measured by echocardiogram at baseline (4 h post-MI), 28 days, and 70 days after treatment (n=5). P, platelets, Antibody, Gevokizumab; IL1-PM, Gevokizumab-attached platelets. *p<0.05, **p<0.01, ***p<0.001, ns, no significance.

FIG. 7: Gevokizumab modification. SDS-PAGE analysis of Gevokizumab (1) and DSPE-PEG-Gevokizumab (2).

FIGS. 8A-8D: TEM characterization of IL1-PM. (A) Representative TEM images showing Gevokizumab-conjugated platelet microparticles (IL1-PM). Platelet marker CD42b and Gevokizumab were detected using gold nanoparticle-labeled secondary antibodies with diameters of 10 nm and 20 nm, respectively. (B) Enlarged image of the red box area in (A). White circles indicate 10 nm gold nanoparticles while yellow arrowheads indicate 20 nm gold nanoparticles. (C) Representative STEM images showing IL1-PM. (D) TEM mapping indicating the presence of Au on the surface of platelets.

FIGS. 9A-9B: Arming of Gevokizumab onto platelets. IL1-PM were labeled with FITC-labeled secondary antibodies and imaged by fluorescent microscope (A) or examined by flow cytometry (B). Scale bar, 10 μm.

FIGS. 10A-10D: Confirming antibody conjugation and detoxification rate. (A) DLS and (B) Zeta potential of platelets before and after DSPE-PEG-Gevokizumab conjugation; (C) Line graph summarizing the adsorption efficiency of Gevokizumab on platelets; (D) The detoxification efficiency of IL1-PM.

FIG. 11: Platelet markers on IL1-PM. Surface marker expression on native (non-conjugated) and antibody-conjugated platelet microparticles (IL1-PM).

FIGS. 12A-12E: Inactivated platelet microparticles can bind to damaged vasculatures. (A) Aggregometry was performed on platelet rich plasma (PRP) mixed with PBS and IL1-PM. (B) P-selectin expression on the surface of platelets: black line, negative control (platelets); red line, IL1-PM; purple line, positive control (platelets with collagen). (C) Control aorta. (D) Healthy aorta incubated with IL1-PM. (E) Denuded aorta incubated with IL1-PM. Scale bar, 10 μm.

FIG. 13: Circulation lifetime of IL1-PM in normal mice. Plasma concentration-time profile after administration of DiD-labeled IL1-PM or normal platelets in normal mice. ns indicates no significant difference.

FIGS. 14A-14D: Toxicity of IL1-PM treatment and quantification of IL-1β and IL-6 concentrations in the heart. (A) Histological assessments of major organs with H&E staining in mice 4 weeks after IL1-PM treatment (×200). Dose-finding study for IL1-PM treatment. (B) Standard curve. (C) The concentrations of IL-1β. (D) Detection of IL-6 in the blood 3 days after treatment. **p<0.01 and ***p<0.001, respectively. NS indicates no significant difference.

FIGS. 15A-15D: Inflammatory response after treatments. The effects of IL1-PM treatment on the number of macrophages (A) and T cells (C). Quantitative analysis of the number of F4/80-positive cells (B) and CD3-positive cells (D). Scale bars, 50 μm, IL1-PM group vs other groups. ns indicates no significant difference.

### DETAILED DESCRIPTION

Embodiments of the present disclosure provide novel compositions and methods for treating cardiac injury using anti-IL-1β platelet microparticles (IL1-PMs) to promote cardiac detoxification and repair after cardiac injury (e.g., myocardial infarction). By including a targeting group to IL-1β blockers to enhance their accumulation in the site of disease, the risk of side effects can be reduced, and therapeutic efficiency can be improved.

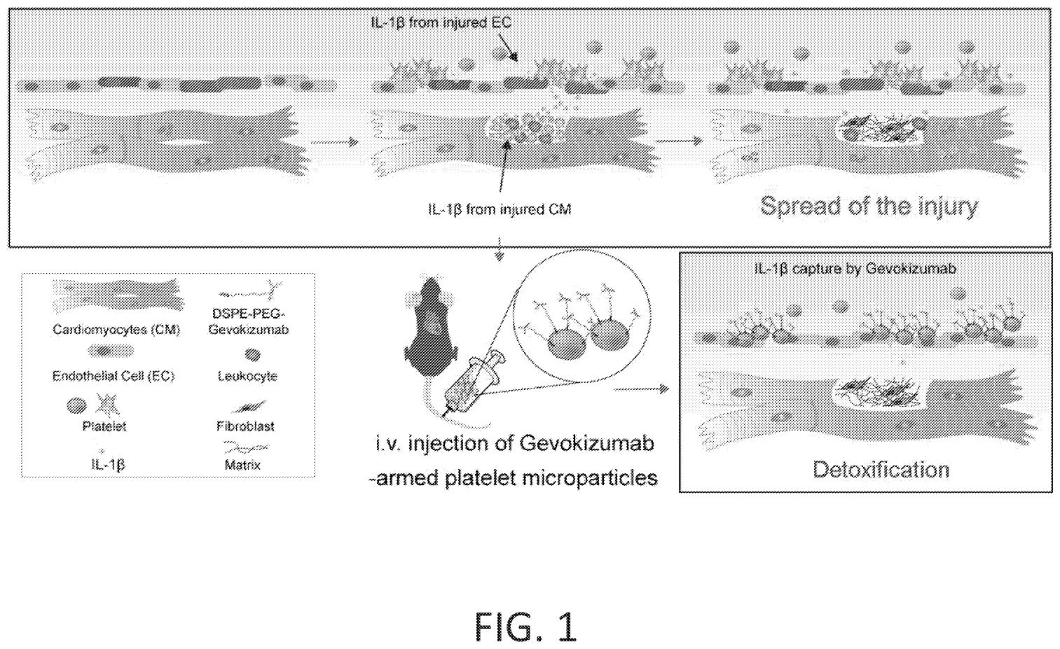

Previous studies demonstrated that it is possible to take advantage of the natural infarct-homing abilities of platelet membranes by using them to decorate stem cells and particles for the targeted repair of injured hearts. A platelet mimicking system was developed that uses anti-IL-1β-neutralizing antibodies. This system functions as an IL-1β decoy that reduces the local inflammatory response in the injured heart (FIG. 1) in a targeted way. In order to capture the IL-1β, a potent monoclonal antibody, Gevokizumab, was used. Gevokizumab (also called XOMA 052 and developed by XOMA Corporation) is an anti-inflammatory agent that has been used in clinical trials to treat acne vulgaris, osteoarthritis, Bechet's uveitis, pyoderma gangrenosum, and Bechet's disease. It was first modified for the binding of platelets using 1, 2-Distearoyl-sn-glycero-3-phosphoethanolamine-Poly(ethylene glycol) (DSPE-PEG) derivatives. Taking advantage of the infarct-homing ability of platelets, the IL-1β decoys of the present disclosure are transported via the circulation to the MI area, where they neutralize IL-1β and, thus, prevent adverse cardiac remodeling and eventual heart failure.

Cardiovascular disease remains as the primary killer in western societies. Stem cell transplantation provides a promising method for cardiac regeneration, but current therapies are limited by inefficient interaction between potentially beneficial cells and the injured tissue which is highly inflammatory. Novel approaches are required to not only bring more “seeds” (therapeutic cells) to their targets, but also to improve the “soil,” the inflammatory post-MI heart. IL-1β plays a key role in triggering the inflammatory cascade in the infarcted myocardium. Thus, in the present disclosure, an anti-IL-1β antibody-platelet conjugate was developed that serves as a cardiac detoxification and anti-inflammatory therapeutic for the treatment of AMI. Platelet microparticles were chosen as antibody carriers because of their innate ability to find cardiac injury. Circulating platelets can bind to vessel lesions through the interaction of GPIb (CD42b) with von Willebrand factor (vWF) which subsequently induces platelet activation in the infarct area. Later on, GPIIb/IIIa activation and P-selectin expression further trigger platelet aggregation.

Previous studies have used a scFv anti-GPIIb/IIIa functionalized PET tracer for the detection of minimal cardiac ischemia through imaging of activated platelets in the infarct area. Bispecific antibodies were synthesized to deliver both anti-inflammatory molecular and peripheral blood mononuclear cells (PBMC) for the treatment of myocardial infarction. These investigations demonstrated that platelets were both promising targets and carriers in the treatment of heart injury. The findings of the present disclosure demonstrate that neutralizing IL-1β protects cardiomyocytes from apoptosis by attenuating caspase-3 activity downstream of IL-1β production and inhibiting the development of ventricular dilation after an AMI.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

The terms “administration of” and “administering” a composition as used herein refers to providing a composition of the present disclosure to a subject in need of treatment. The compositions of the present disclosure may be administered by intramuscular, intraperitoneal, intravenous, ICV, intracisternal injection or infusion, subcutaneous injection, or implant, surgical intervention, or similar routes of administration, and may be formulated, alone or together, in suitable dosage unit formulations containing conventional non-toxic pharmaceutically acceptable carriers, adjuvants and vehicles appropriate for each route of administration.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing, e.g., microparticles of the present disclosure and a pharmaceutically acceptable carrier and/or excipient. When microparticles of the present disclosure are used contemporaneously with one or more other drugs, a pharmaceutical composition containing such other drugs in addition to the microparticles of the present disclosure are contemplated. Accordingly, the pharmaceutical compositions of the present disclosure include those that also contain one or more other active ingredients, in addition to microparticles of the present disclosure. The weight ratio of the microparticles of the present disclosure may be varied and will depend upon the effective dose of each ingredient. Generally, an effective dose of each will be used. Combinations of microparticles of the present disclosure and other active ingredients will generally also be within the aforementioned range, but in each case, an effective dose of each active ingredient should be used. In such combinations the microparticles of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition, and/or to improve/enhance one or more aspects of a subject's physical health. The compositions can be formulated according to known methods for preparing pharmaceutically useful compositions (e.g., microparticle preparation). Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “effective amount” generally means that amount of a drug or pharmaceutical agent that will elicit the biological or medical response of a tissue, system, animal or human that is being sought, for instance, by a researcher or clinician. Furthermore, the term “therapeutically effective amount” generally means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “combination” and derivatives thereof, as used herein, generally means either, simultaneous administration or any manner of separate sequential administration of a therapeutically effective amount of Compound A, or a pharmaceutically acceptable salt thereof, and Compound B or a pharmaceutically acceptable salt thereof, in the same composition or different compositions. If the administration is not simultaneous, the compounds are administered in a close time proximity to each other. Furthermore, it does not matter if the compounds are administered in the same dosage form (e.g., one compound may be administered topically and the other compound may be administered orally).

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2).

“Antibody fragment” as used herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds to.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9: 133-139 (1995), and MacCallum, J Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, cardiovascular biology, genetics and biochemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Compositions and Methods

The present disclosure provides compositions and methods relating to the use of platelet microparticles to deliver therapeutic antibodies. In particular, the present disclosure provides novel compositions and methods for treating cardiac injury using anti-IL-1β platelet microparticles (IL1-PMs) to promote cardiac detoxification and repair after cardiac injury (e.g., myocardial infarction).

In accordance with these embodiments, the present disclosure includes a platelet-derived microparticle comprising a linker moiety and at least one therapeutic antibody. The microparticles of the present disclosure were developed as antibody carriers because of their ability to find cardiac injury (e.g., sites of inflammation after a myocardial infarction). For example, circulating platelets can bind to vessel lesions through the interaction of GPIb (CD42b) with von Willebrand factor (vWF), which subsequently induces platelet activation in the infarct area. Subsequently, GPIIb/IIIa activation and P-selectin expression further trigger platelet aggregation. In this manner, the microparticles of the present disclosure are derived from inactivated platelets and used for the targeted delivery of a therapeutic cargo to an injury site. Generally, circulating unactivated platelets are biconvex discoid structures, about 2-3 μm in greatest diameter; activated platelets have cell membrane projections covering their surface. In some embodiments, the microparticles are derived from inactivated platelets.

In some embodiments, a linker moiety is used to attach one or more therapeutic agents to the microparticles in order to treat an injury site. The linker moiety can be any biocompatible linker known in the art based on the present disclosure, including, but not limited to, a linker comprising phosphatidylethanolamine (PE). In some embodiments, the same linker moiety is used to attach different therapeutic agents to the microparticles. In other embodiments, different linkers are used to attach different therapeutic agents to the microparticles. In some embodiments, the linker moiety is functionally coupled to the surface of the microparticle. In some embodiments, the linker moiety is functionally coupled to the surface of the microparticle via a lipophilic headgroup. In some embodiments, the linker moiety comprises 1,2-Distearoyl-sn-glycero-3-phosphorylethanolamine (DSPE). In some embodiments, the linker moiety comprises a PEG polymer. In some embodiments, the linker moiety comprises DSPE and a PEG polymer. In some embodiments, the PEG polymer is at least 5 kDa. In some embodiments, the linker moiety comprises an NHS-terminated DSPE-PEG polymer, or any derivatives or variants thereof.

In some embodiments, the linker moiety facilitates the attachment of a therapeutic antibody to the platelet-derived microparticles. In some embodiments, the at least one therapeutic antibody is covalently bound to the linker moiety. Other means for attaching a therapeutic antibody to the microparticles can also be used, provided that the means for attachments is suitable for coupling to a platelet-derived microparticle (e.g., a lipid-based attachment, e.g., via a membrane).

In some embodiments, the at least one therapeutic antibody neutralizes at least one aspect of an immune response. In some embodiments, the at least one therapeutic antibody is a monoclonal antibody. In other embodiments, the antibody is a derivative of a monoclonal antibody (e.g., Fab fragment or similar antigen binding domain). In some embodiments, the antibody is a bi-specific antibody, or a derivative thereof. In some embodiments, the at least one therapeutic antibody binds to IL-1β. In some embodiments, the at least one therapeutic antibody binds to IL-1β and another cytokine. In some embodiments, the at least one antibody is Gevokizumab, Canakinumab, and a derivative, variant, or a combination thereof. In other embodiments, the antibody targets at least one of IL-1α, IL-16, IL-18, and TNF-α, or any combinations thereof. As described above, the microparticles of the present disclosure can include one or more antibodies targeting IL-1β, IL-1α, IL-16, IL-18, and TNF-α, or any combinations thereof. In some embodiments, these antibodies can include, but are not limited to, IL-1 blockers such as Anakinra (a recombinant human receptor antagonist), Canakinumab (a human monoclonal antibody), and Rilonacept (a soluble IL-1R1-AcP), as well as antibodies that target TNF-α, including but not limited to infliximab, etanercept and adalimumab.

Embodiments of the present disclosure also include a composition comprising a plurality of microparticles described herein, and at least one pharmaceutically acceptable carrier or excipient. In accordance with these embodiments, the compositions of the present disclosure can include the plurality of microparticles comprising at least one therapeutic antibody, as well as one or more other components, including but not limited to, a pharmaceutical carrier, excipient, adjuvant, and/or pH buffer. In some embodiments, a pharmaceutically acceptable carrier includes diluents, adjuvants, and vehicles, as well as implant carriers, and inert, non-toxic solid or liquid fillers, diluents, or encapsulating material.

In some embodiments, the compositions of the present disclosure include a second therapeutic agent, in addition to the plurality of microparticles comprising at least one therapeutic antibody. Examples of additional therapeutic agents that can be included in the compositions of the present disclosure include, but are not limited to, beta-blockers, ACE inhibitors, anti-inflammatory agents, and the like. In some embodiments, the additional therapeutic agent(s) can enhance the efficacy of the plurality of microparticles. In some embodiments, the therapeutic agents include antithrombotic drugs such as antiplatelet drugs (e.g., aspirin, clopidogrel, and glycoprotein IIb/IIIa receptor antagonists), which minimize blood coagulation. In some embodiments, antithrombotic agents may interfere with the ability of the PMs to target the site of inflammation, thus, co-treatment with anti-thrombotic agents may be counterproductive. However, secretion and growth factors (e.g., bFGF, HGF, VEGF) from stem cells may be benefit to an injured heart when administered with IL-1 PMs owing to their therapeutic effect.

Embodiments of the present disclosure also include a method for treating a subject that has suffered a cardiac event. In accordance with these embodiments, the method includes administering any of the microparticles and/or compositions described herein to a subject in need thereof (e.g., a subject that has suffered a myocardial infarction). In some embodiments, the composition is administered intravenously, subcutaneously, intramuscularly, or by surgical intervention.

In accordance with the various compositions and methods of the present disclosure, embodiments described herein provide dosage forms, formulations, and methods that confer advantages and/or beneficial pharmacokinetic profiles. A composition of the disclosure can be utilized in dosage forms that confer an efficacious response in a subject. An efficacious response may be obtained by administering a dosage once, twice a day, or three times a day, or more administration, each dosage comprising the compositions/microparticles present in an amount sufficient to provide the required concentration or dose of the composition/microparticles to treat a disease disclosed herein (e.g., a myocardial infarction).

Embodiments of the present disclosure relate to a dosage form comprising one or more compositions of the present disclosure that can provide peak plasma concentrations of the composition of between about 0.001 to 2 mg/ml, 0001 to 1 mg/ml, 0.0002 to 2 mg/ml, 0.005 to 2 mg/ml, 001 to 2 mg/ml, 0.05 to 2 mg/ml, 0.001 to 0.5 mg/ml, 0.002 to 1 mg/ml, 0.005 to 1 mg/ml, 0.01 to 1 mg/ml, 005 to 1 mg/ml, or 0.1 to 1 mg/ml. The disclosure also provides a formulation or dosage form comprising one or more compositions of the present disclosure that provides an elimination ti_{/2} of 0.5 to 20 h, 0.5 to 15 h, 0.5 to 10 h, 0.5 to 6 h, 1 to 20 h, 1 to 15 h, 1 to 10 h, or 1 to 6 h.

A subject may be treated with a composition of the present disclosure or composition or unit dosage thereof on substantially any desired schedule (e.g., after a myocardial infarction). They may be administered one or more times per day, in particular 1 or 2 times per day, once per week, once a month or continuously. However, a subject may be treated less frequently, such as every other day or once a week, or more frequently. A composition or composition may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, 2 weeks to 24 months, or for more than 24 months, periodically or continuously.

A beneficial pharmacokinetic profile can be obtained by the administration of a formulation or dosage form suitable for once, twice, or three times a day administration, or as often as needed. The required dose of a composition of the disclosure administered once twice, three times or more daily is about 0.01 to 3000 mg/kg, 0.01 to 2000 mg/kg, 0.5 to 2000 mg/kg, about 0.5 to 1000 mg/kg, 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 50 mg/kg, 0.1 to 20 mg/kg, 0.1 to 10 mg/kg, 0.1 to 6 mg/kg, 0.1 to 5 mg/kg, 0.1 to 3 mg/kg, 0.1 to 2 mg/kg, 0.1 to 1 mg/kg, 1 to 1000 mg/kg, 1 to 500 mg/kg, 1 to 400 mg/kg, 1 to 300 mg/kg, 1 to 200 mg/kg, 1 to 100 mg/kg, 1 to 50 mg/kg, 1 to 20 mg/kg, 1 to 10 mg/kg, 1 to 6 mg/kg, 1 to 5 mg/kg, or 1 to 3 mg/kg, or 1 to 2.5 mg/kg, or less than or about 10 mg/kg, 5 mg/kg, 2.5 mg/kg, 1 mg/kg, or 0.5 mg/kg twice daily or less.

The present disclosure also contemplates a formulation or dosage form comprising amounts of one or more composition of the disclosure that results in therapeutically effective amounts of the composition over a dosing period, in particular a 24 h dosing period. The therapeutically effective amounts of a composition of the disclosure are between about 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 75 mg/kg, 0.1 to 50 mg/kg, 0.1 to 25 mg/kg, 0.1 to 20 mg/kg, 0.1 to 15 mg/kg, 0.1 to 10 mg/kg, 0.1 to 9 mg/kg, 0.1 to 8 mg/kg, 0.1 to 7 mg/kg, 0.1 to 6 mg/kg, 0.1 to 5 mg/kg, 0.1 to 4 mg/kg, 0.1 to 3 mg/kg, 0.1 to 2 mg/kg, or 0.1 to 1 mg/kg.

A medicament or treatment of the disclosure may comprise a unit dosage of at least one composition of the disclosure to provide therapeutic effects. A “unit dosage or “dosage unit” refers to a unitary (e.g., a single dose), which is capable of being administered to a patient, and which may be readily handled and packed, remaining as a physically and chemically stable unit dose comprising either the active agents as such or a mixture with one or more solid or liquid pharmaceutical excipients, carriers, or vehicles.

### 3. Materials and Methods

Gevokizumab-conjugated platelet microparticles (IL1-PMs). DSPE can bind with the membranes of cells, liposomes, and platelets. The surface of the platelets was functionalized with anti-IL-1β antibodies. The inactivated platelets were isolated according to previous works. In brief, whole blood was collected from the C57BL/6 mice (non-terminal collection from the orbital sinus or saphenous vein; 20 mice were used) into a plastic syringe containing 1.0 mL citrate-phosphatedextrose (16 mM citric acid, 90 mM sodium citrate, 16 mM NaH2PO4, 142 mM dextrose, pH 7.4) and centrifuged at 100 g for 20 min at room temperature. The platelet-rich plasma (PRP) was transferred to a separate tube using a transfer pipette (wide orifice), and PGE1 was added to each tube at a final concentration of 1 μM. Platelets were isolated from the PRP via centrifugation at 800 g for 10 min. The plasma was discarded, and the platelets were resuspended carefully in Tyrode's buffer (134 mM NaCl, 12 mM NaHCO₃, 2.9 mM KCl, 0.34 mM Na2HPO4, 1 mM MgCl2, 10 mM HEPES, pH 7.4) or PBS with PGE1 added at 1 μM.

Then 10⁸ platelets were dispersed in 0.5 ml of PBS buffer with 1×10⁻³ M of ethylenediaminetetraacetic acid (EDTA) and 2×10⁻⁶ M of prostaglandin E1 (PGE1) and then, DSPE-PEG-Gevokizumab was added at different concentrations. The mixture was stirred for 3 h. Unconjugated DSPE-PEG-Gevokizumab was removed by centrifugation at 800×g for 10 min. The IL1-PM were then washed twice with PBS using centrifugation at 800×g for 10 min.

Gevokizumab (also called XOMA 052) was obtained from Creative Biolabs (NY, USA). DSPE-PEG-NHS (MW=5K) was purchased from NANOCS. Mouse IgG total ELISA Kit was obtained from Fisher Scientific. Anti-mouse CD45, caspase-1, caspase-3, F4/80+, CD3 antibodies, goat anti-mouse and anti-rabbit IgG secondary antibodies labeled with Alexa Fluor 488 and Texas Red were purchased from Abcam. Anti-apoptosis-associated speck-like protein containing a caspase recruitment domain (ASC) antibody, in situ cell death detection TMR red (TUNEL) kit, trichrome stain (Masson's Trichrome) kit, and anti-Mouse IgG (whole molecule) gold antibody produced in goat were purchased from Sigma-Adrich. Sulfo-Cyanine5.5 NHS ester was obtained from Lumiprobe. Caspase-1 assay kit, caspase-3 Assay Kit (Promega™ CaspACE™ Assay System) and Raybiotech IncSupplier Diversity Partner MOUSE CYTOKINE ARRAY C3 (4) were purchased from Fisher Scientific.

Anti-IL-1β antibodies conjugated with DSPE-PEG-NHS. Anti-IL-1β antibodies, Gevokizumab, were first reacted with DSPE-PEG-NHS through —NH₂ and NHS acylation reactions. Equimolar antibodies and DSPE-PEG-NHS were mixed together and reacted at 4° C. for 24 h. Then, the unreacted DSPE-PEG-NHS was removed by centrifugation using an Amicon Ultra-0.5 Filter (100 kDa). Successful conjugation was confirmed by SDS-PAGE.

Quantification of antibodies on platelets. The IL1-PM were resuspended in 100 μL of deionized water and ultrasonicated to lyse the platelets and release the DSPE-PEG-Gevokizumab. The quantity of antibodies conjugated to the platelets was measured using ELISA. The numbers of antibodies per platelet were calculated by the equation of:

N(antibody)=m_{antibody}/Mw*NA/Number of platelets

Determining antibody arming efficiency on platelets. IL1-PMs were first incubated with anti-CD42b antibodies (species: rabbit) overnight. Then gold nanoparticle-labeled goat anti-mouse IgG antibodies (20 nm) and goat anti-rabbit IgG antibodies (20 nm) were used to bind anti-IL-1β and anti-CD42b primary antibodies, respectively. In addition, FITC-labeled goat anti-mouse IgG antibodies were also used to confirm the presence of anti-IL-1β on the surface of platelets. The free IgG antibodies were removed though centrifugation (10 min at 800×g). The prepared samples were examined using TEM and fluorescence microscopy.

Studies on platelet activation. The expression of P-selectin were determined using flow cytometry. Freshly prepared platelets were activated with collagen as a positive control. Then positive control and IL1-PM samples were incubated with PE-labeled anti-P-selectin (12-0626-80, Thermal Fisher) overnight. Samples were washed with PBS for three times.

Mouse model of myocardial infarction. All animal work was compliant with the Institutional Animal Care and Use Committee (IACUC) of North Carolina State University. MI model were constructed according to previous work.

Infarct-homing ability and biodistribution of IL1-PM. IL1-PM@Cy5.5 or antibody@Cy5.5 were intravenously injected into MI mice or normal (non-MI) mice (n=3) at a dose of 2 mg antibodies per kg body weight. Animals were imaged after various time intervals for biodistribution analysis. Circulation lifetime of IL1-PM and platelets in normal mice was studied. About 10⁸ DiD-labeled IL1-PMs or platelets were i.v. injected into normal mice. At various time points, blood was drawn and measured for fluorescence intensity at 670 nm for evaluation of circulation life time of the agents.

Cytokine array analysis of systemic inflammatory cytokines. Plasma levels of cytokines were measured 3 days after treatments using a Raybiotech IncSupplier Diversity Partner MOUSE CYTOKINE ARRAY C3 (4), according to the manufacturer's instructions. In addition, the plasma levels of IL-6 were tested using ELISA kit.

Statistical analysis. All experiments were performed independently at least three times. Results are shown as means±standard deviation. Comparisons between any two groups were performed using the two-tailed, unpaired Student's t-test. Comparisons among more than two groups were performed using one-way ANOVA, followed by the post hoc Bonferroni test. Single, double, and triple asterisks represent p<0.05, 0.01, and 0.001, respectively; p<0.05 was considered statistically significant.

Denuded rat aorta binding assay. To examine the binding of IL1-PM onto injured (denuded) vascular walls, aortas from C57BL/6 mice were dissected and surgically scraped on their luminal side with forceps, to remove the endothelial layer. Both denuded or control aortas were incubated with DiO-labeled IL1-PM for 5 min. After PBS rinses, the samples were subjected to fluorescence microscopy examination for IL1-PM binding.

Aggregation assay. To determine if IL1-PM induced any adverse pro-thrombotic effects, 450 μL of platelet poor plasma (PRP) was collected and added to glass cuvettes for aggregometry. Aggregometry was performed using a commercial optical (light transmission) aggregometer (Chrono-log 700 manual, Chrono-log Corp.). The following analyses were performed: (1) freshly prepared PRP with PBS; (2) freshly prepared PRP with IL1-PM.

Animal experiments. All animal work was compliant with the Institutional Animal Care and Use Committee (IACUC) of North Carolina State University. MI mice were randomized into four treatment groups (n=10 mice per group): 1) i.v. injection of 100 μL PBS; 2) i.v. injection of 10⁸ bare platelets in 100 μL PBS; 3) i.v. injection of anti-IL-1β antibodies at a dose of 2 mg antibodies/kg mouse body weight; and 4) i.v. injection of IL1-PM at a dose of 2 mg antibodies/kg mouse body weight. Five out of ten mice were sacrificed and the blood and heart tissues were harvested after 3 days of treatment and used for cytokine, western blot, and ELISA analysis. The transthoracic echocardiography procedure was performed with a Philips CX30 ultrasound system, coupled with a L15 high-frequency probe, by a cardiologist blinded to animal group allocation. All animals inhaled a 1.5% isoflurane-oxygen anesthesia mixture in the supine position at the 4-hr, 28 day and 70 day time points (n=5 mice per group). Hearts were imaged in 2D in long-axis views at the level of the greatest left ventricular (LV) diameter. Ejection fraction (EF) was determined by measurements from views taken from the infarcted area. Left ventricular end diastolic volume (LVEDV) and end systolic volume (LVESV) were measured. LVEF was determined by measurement from views taken from the infarcted area. Finally, animals were sacrificed 70 days after injection and hearts were harvested and frozen in OCT compound. Specimens were sectioned at 10 μm thicknesses from the apex to the ligation level with 100 μm intervals. Masson's trichrome staining was performed as described by the manufacturer's instructions. Images were acquired with a PathScan Enabler IV slide scanner (Advanced Imaging Concepts, Princeton, N.J.). From the Masson's trichrome stained images, morphometric parameters, including viable myocardium, scar size, and infarct thicknesses were measured in each section with NIH ImageJ software. The percentage of viable myocardium as a fraction of the scar area (infarcted size) was quantified. Three selected sections were quantified for each animal.

Western blot and ELISA analysis. The hearts harvested after the 3^{rd} day of treatments were homogenized in RIPA buffer and supplemented with a protease inhibitor cocktail. The obtained solutions were centrifuged at 16,000×g for 20 min to remove non-homogenized tissue. Then, the total protein concentration was measured using a BCA Protein Assay Kit. After that, the CD45, caspase-1, and caspase-3 were analyzed using western blot. In addition, Caspase-1 Assay Kit (Fluorometric) was used for detecting the activity of caspases that recognized the sequence YVAD using the fluorometric method. Caspase-3 substrate Ac-DEVD-pNA was applied to detect the caspase-3 activities in the heart using colorimetric analysis kits according to the manufacturer's instructions. Furthermore, the change of IL-1β was detected using a mouse anti-IL-1β kit, according to the manufacturer's instructions.

Immunohistochemistry assessment. Heart cryosections were fixed with 4% paraformaldehyde in PBS for 30 min, permeabilized, and blocked with Protein Block Solution (DAKO) containing 0.1% saponin for 1 h, at room temperature. The cryosections were then immunoassayed with anti-ASC antibodies to detect the presence of inflammasome, and with TUNEL to quantify cardiomyocyte apoptosis. In addition, anti-mouse alpha sarcomeric actin antibodies were used to co-stain and identify cardiomyocytes. Furthermore, anti-CD3 (T cells) and F4/80 (macrophages) antibodies were used to test the immune response in the heart. For IHC study, hearts were cryo-sectioned at 10 μm thickness from the apex to the ligation level with 100 μm intervals for immunohistochemistry. Four slides were stained for each animal and 4 randomly selected fields from each slide (n=4) were analyzed with NIH ImageJ software. The red, green, and blue channels were split (RGB), and the integrated densities of the green signal were calculated using the software.

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

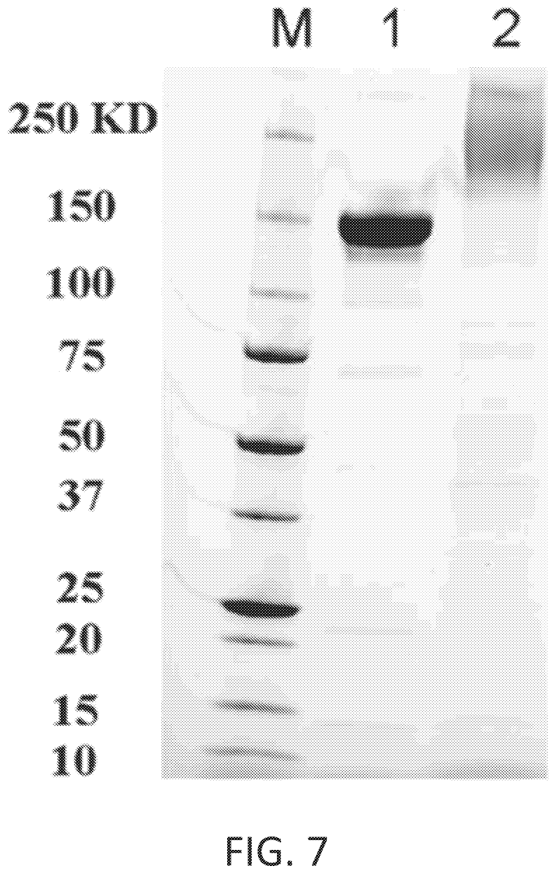

Fabrication of IL1-PMs. In order to create the platelet-Gevokizumab linkage, NHS-terminated DSPE-PEG polymers were bound to the platelet membranes. The Gevokizumab antibodies were then covalently bound to the NHS ends. SDS-PAGE was used to demonstrate the binding of Gevokizumab to the DSPE-PEG polymers (FIG. 7). Both conjugated and non-conjugated antibodies were detected. The higher molecular weight (MW) of the conjugated antibodies made them run down the SDS-PAGE gel at a slower pace than the non-conjugated antibodies. Once the coupling of DSPE-PEG and Gevokizumab was confirmed, Gevokizumab-decorated platelets were built by mixing the coupled DSPE-PEG-Gevokizumab with platelets to form anti-IL-1β platelet microparticles (IL1-PM). Inactivated murine platelets were isolated as previously described. To prove the successful conjugation of Gevokizumab with platelets, gold nanoparticles-labeled secondary antibodies were first used to detect the Gevokizumab decorating on platelet surface. IL1-PM were incubated with anti-CD42b antibodies (species: rabbit) overnight. Then gold nanoparticle-labeled goat anti-mouse IgG antibodies (20 nm) and goat anti-rabbit IgG antibodies (20 nm) were used to bind anti-IL-1β and anti-CD42b primary antibodies, respectively.

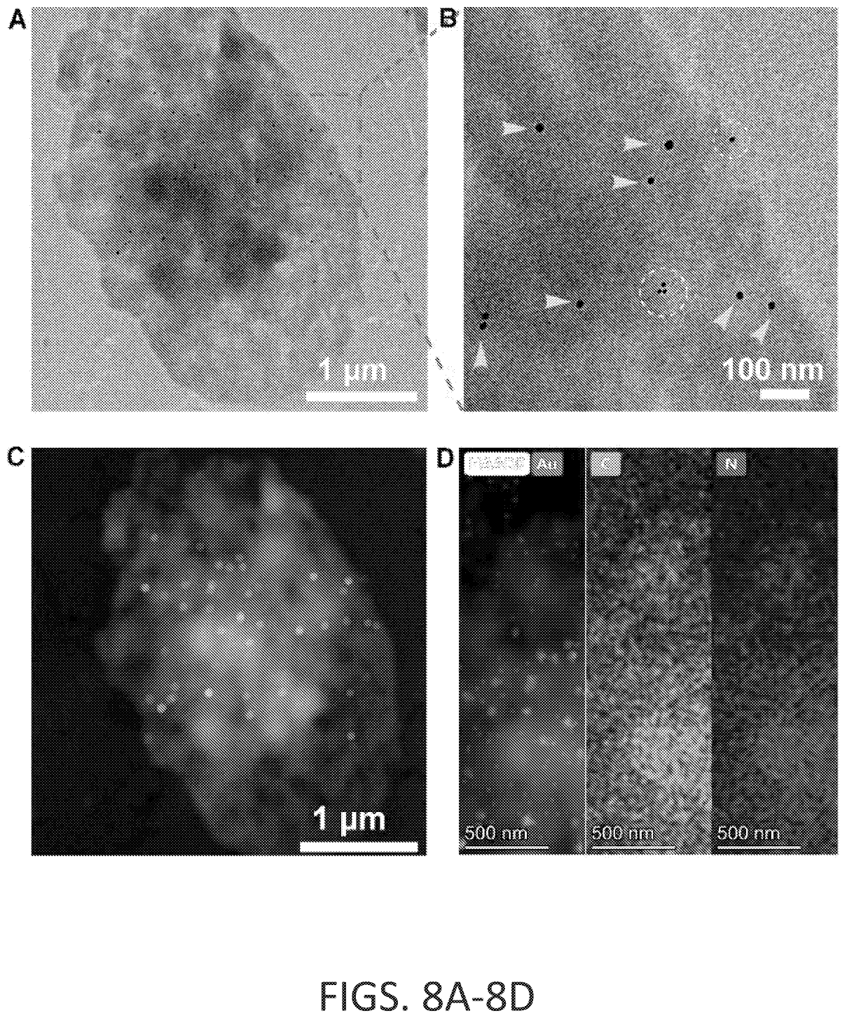

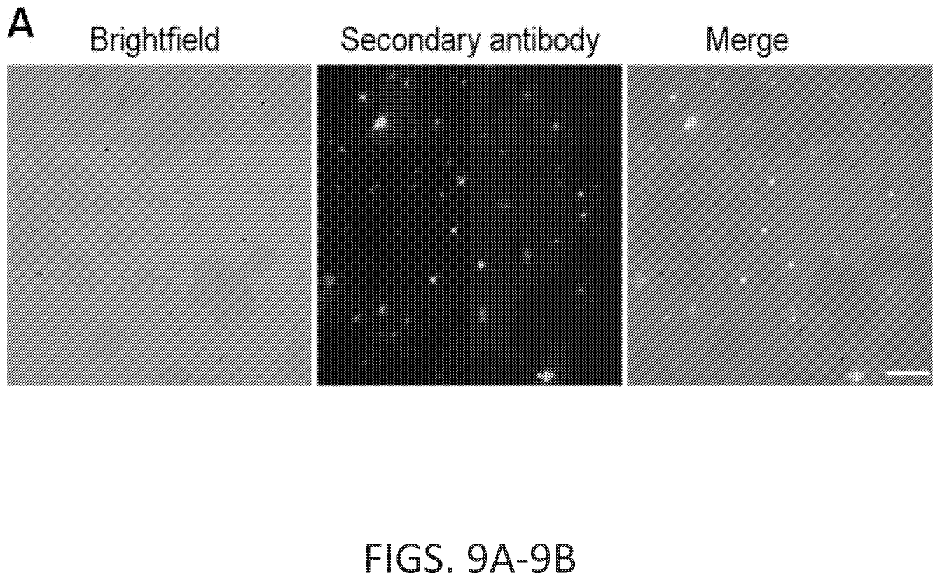

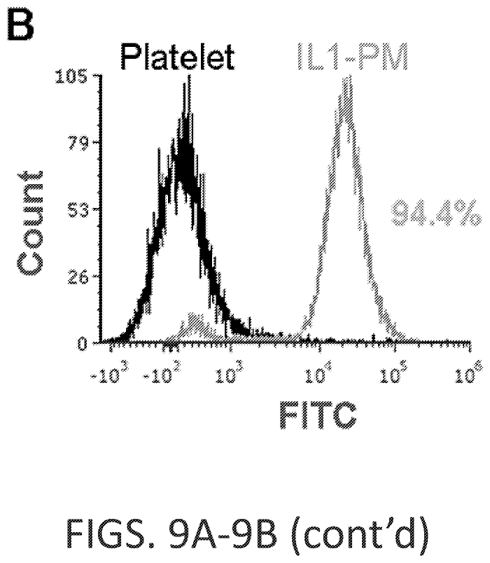

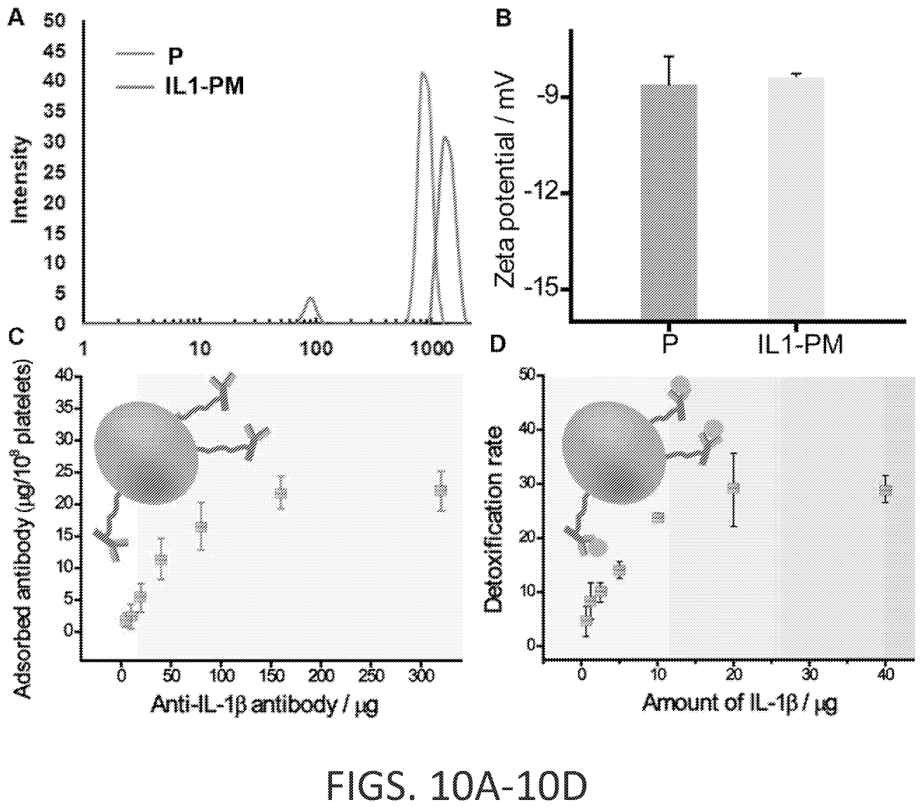

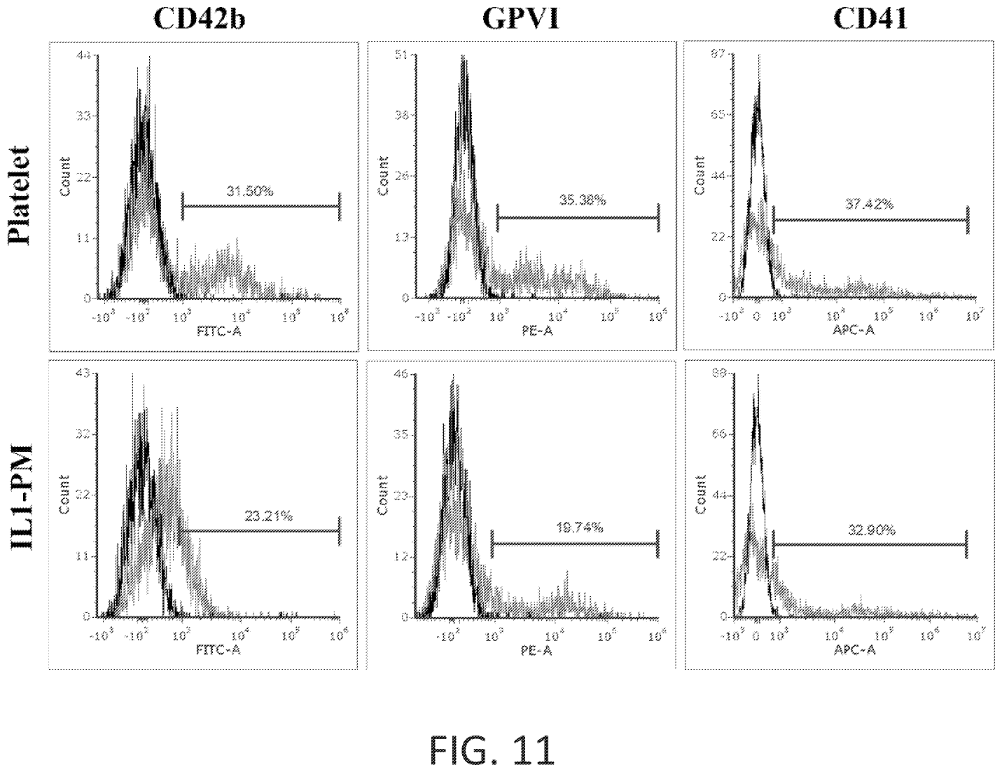

As shown in transmission electron microscope (TEM) images (FIG. 8), both 20 nm and 10 nm gold nanoparticles were detected on the surface of platelets. The presence of 20 nm gold nanoparticles indicated anti-IL-1β antibodies were conjugated onto platelets while the introduction of 10 nm gold nanoparticles confirmed the microparticles were actually derived from platelets (CD42b as a common platelet maker). Furthermore, scanning TEM (STEM) and energy dispersive X-ray (EDX) mapping results confirmed the attached nanoparticles were gold nanoparticles. In addition, fluorescence-labeled secondary antibodies were also used to detect Gevokizumab. Fluorescence microscopy and flow cytometry results showed that most of the platelets were modified by Gevokizumab with high arming efficiency (FIG. 9). Then, the hydrodynamic radius and zeta potential of the platelets were studied before and after Gevokizumab decoration. The dynamic light scattering (DLS) results showed a slight increase in particle size after Gevokizumab conjugation. A number of much smaller particles were also detected, and were mainly attributed to that part of platelets decomposed as the insertion of DSPE-PEG-Gevokizumab (FIG. 10A). In contrast, there was no significant change in the zeta potential (FIG. 10B). Also, the binding ratio of Gevokizumab to platelets was measured. Enzyme-linked immunosorbent assays (ELISA) revealed that about 20 μg of Gevokizumab antibodies were conjugated to 10⁸ platelets (roughly 8.3×10⁵ antibodies per platelet) (FIG. 10C). Despite the decoration of the platelet surface, expressions of platelet surface markers CD41, GPVI, and CD42b were not changed before and after antibody modification (FIG. 11), which confirmed the preservation of the integrity of the platelet membranes. Overall, linking anti-IL-1β antibodies to platelets had a slight effect on the platelets. To examine whether the anti-IL-1β platelet microparticles could capture IL-1β, different concentrations of fluorescence-labeled IL-1β were incubated with IL1-PM. As described herein, the maximum detoxification rate that reduced the amount of IL-1β reached up to 28%.

### Example 2

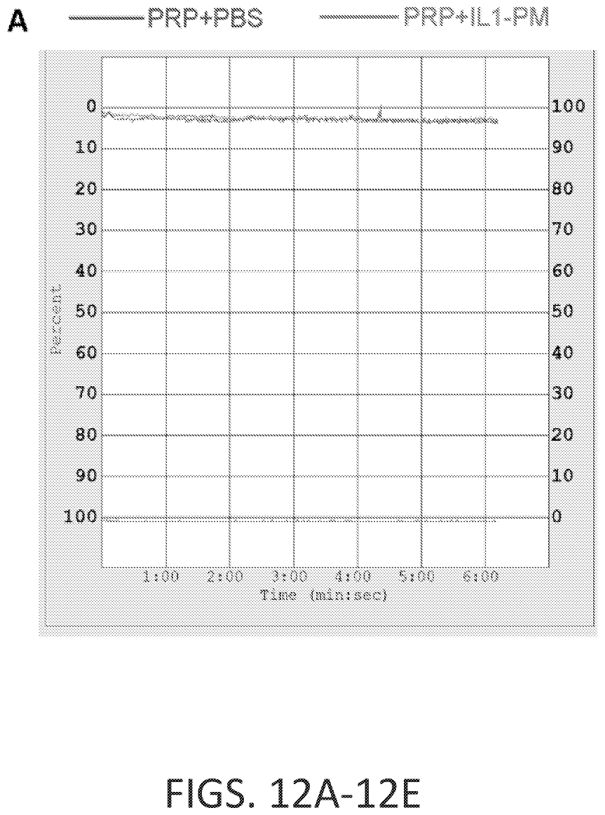

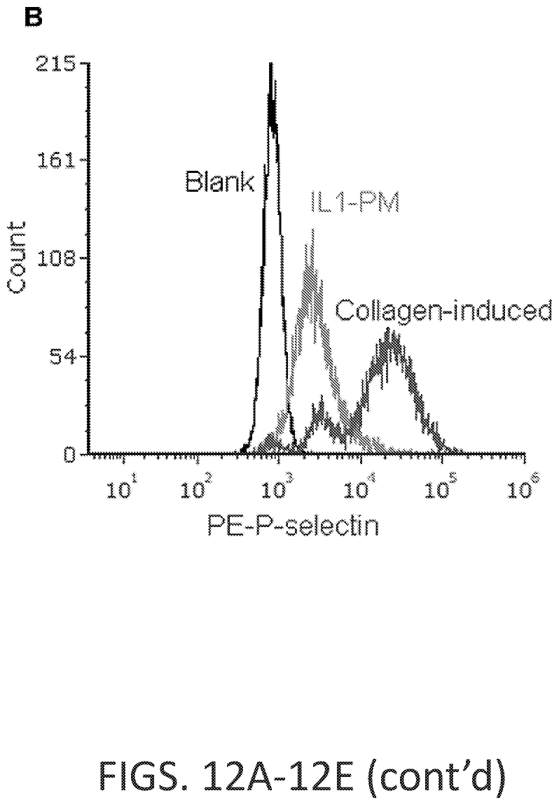

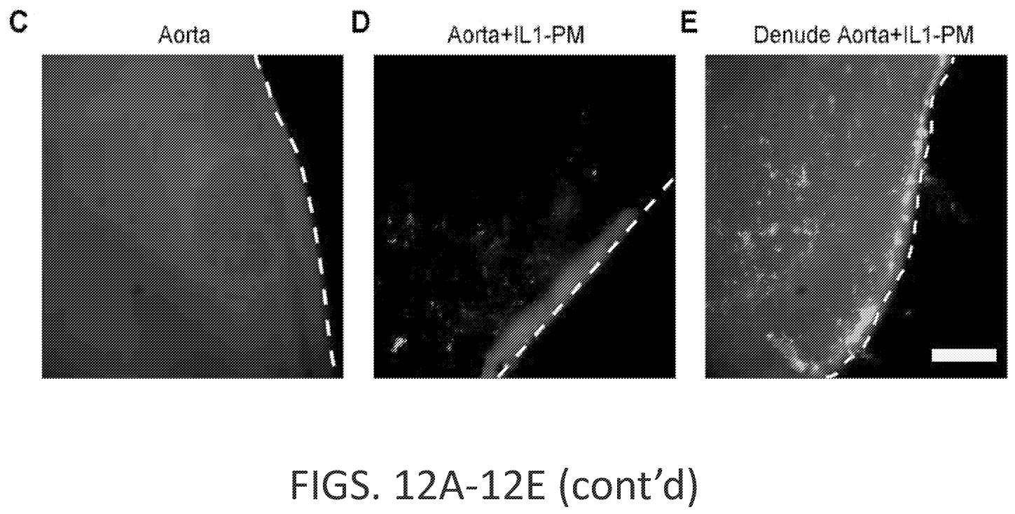

Activity and biding ability of IL1-PM. Activated platelets tend to slowly degranulate and secrete their cytosolic and granule contents. Additionally, such platelets, when introduced in vivo, is rapidly cleared by macrophagic and reticulo-endothelial activity. To see if the platelets would be activated in the process of isolation and modification, platelet aggregometry was carried out and the results confirmed the inactivation of platelets after modification (FIG. 12A). In addition, the expression of P-selectin (a maker of platelet activation) was also detected using flow cytometry and the result was consistent with the results from platelet aggregometry (FIG. 12B). The first contact between circulating platelets and the vessel wall lesion (platelet tethering) was established by an interaction of the platelet receptor for von Willebrand factor (vWF) (GPIb-V-IX) with collagen-immobilized vWF. Direct GPVI-collagen interaction is important for initial platelet tethering and subsequent stable platelet adhesion and aggregation at sites of arterial injury. These data confirmed that IL1-PM preserved the CD42b (GPIb); therefore, inactivated platelets should have the capacity of binding to the injured vessel as long as the binding molecules are present at the surface. Using co-culture experiment, it was confirmed that inactivated IL1-PM possesses the capacity to bind to denoted aorta (FIG. 12C-E).

### Example 3

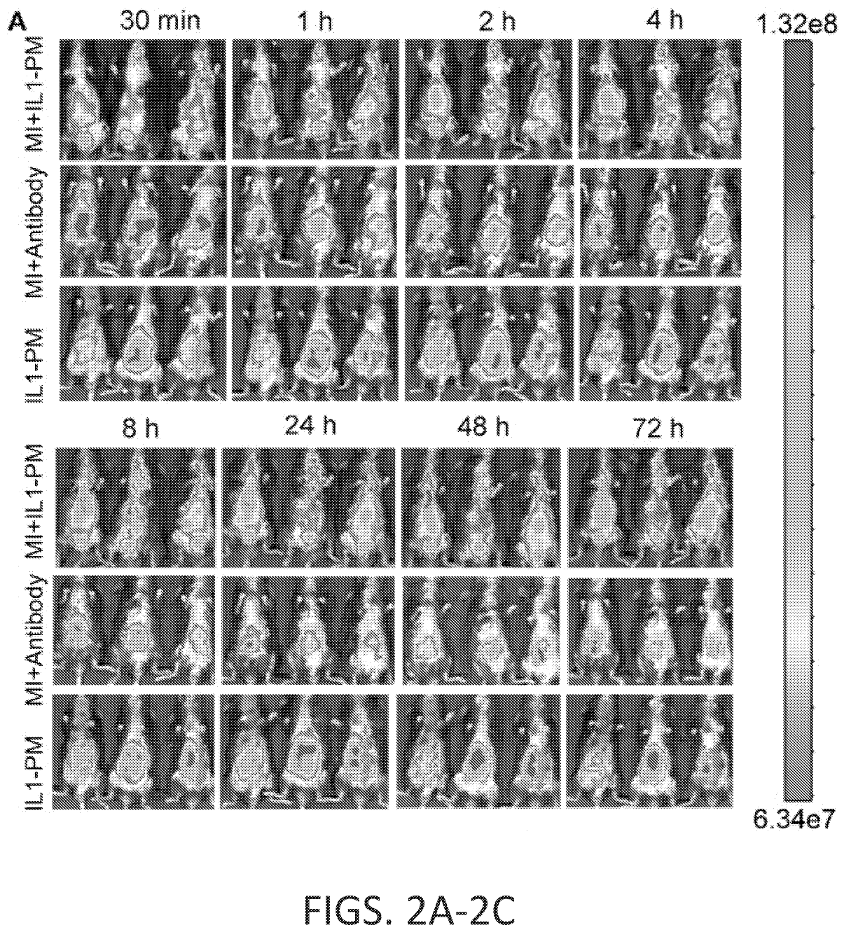

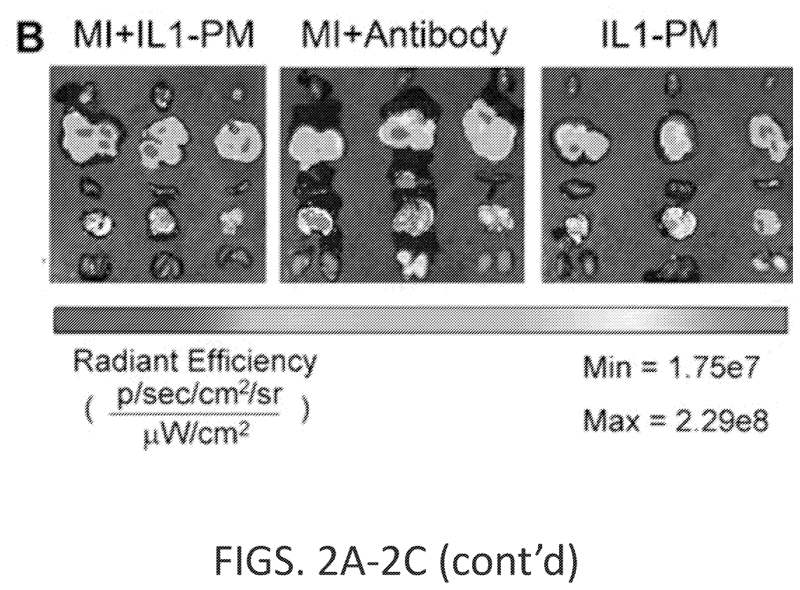

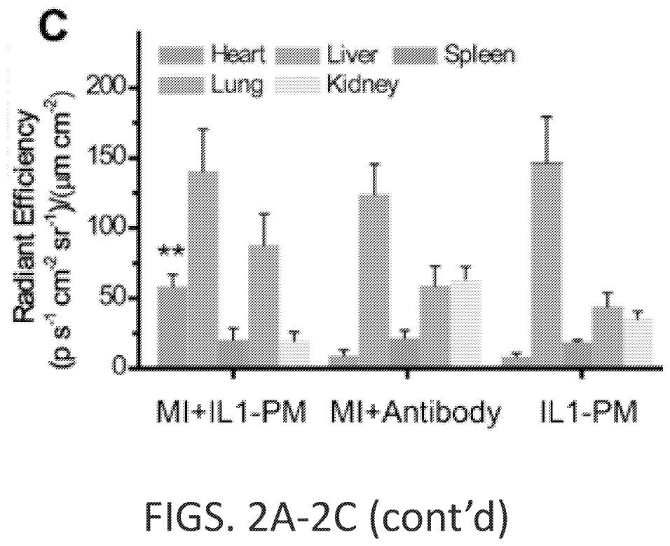

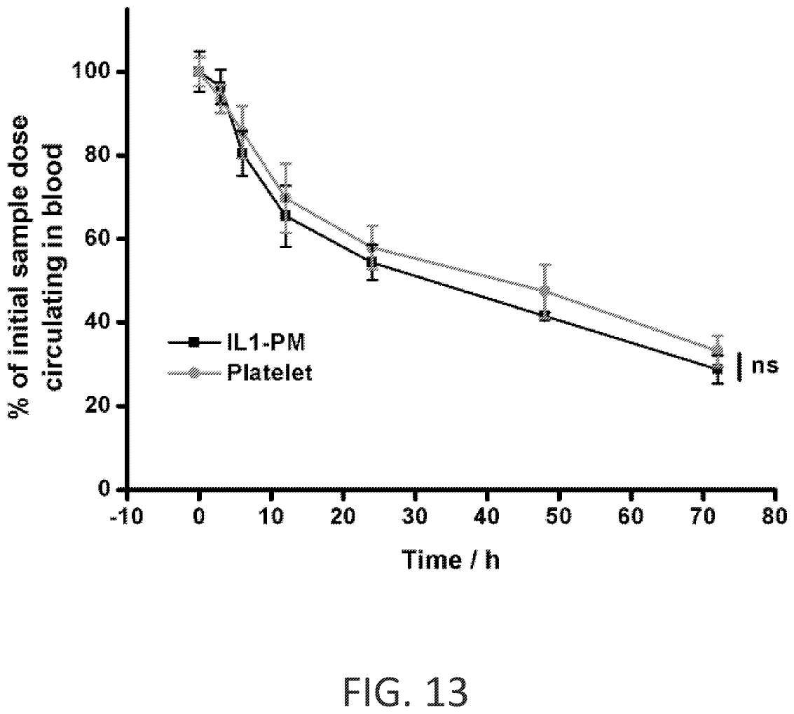

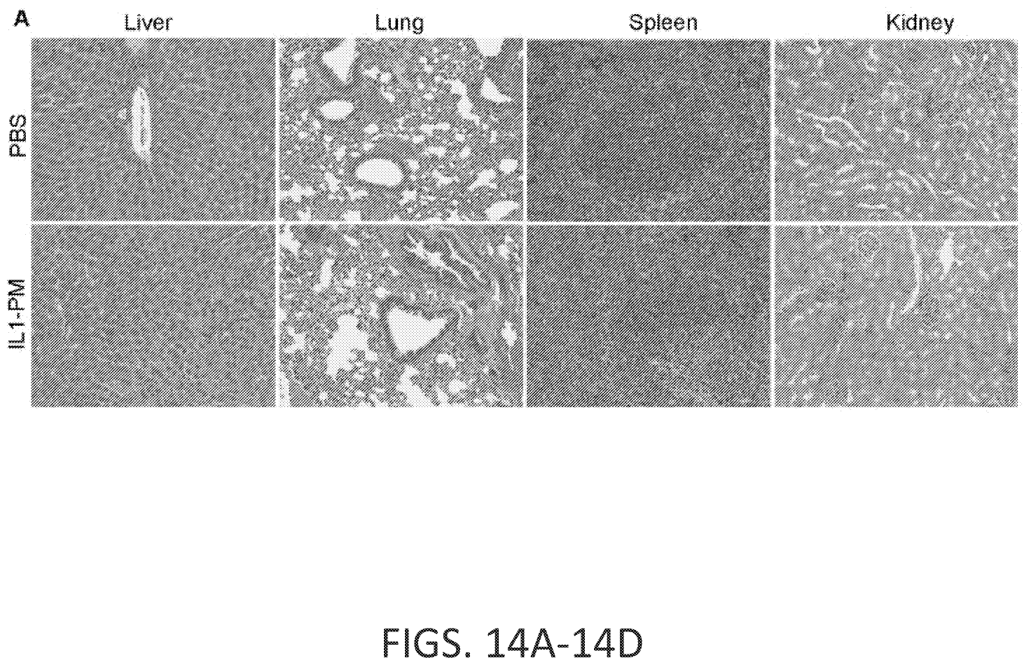

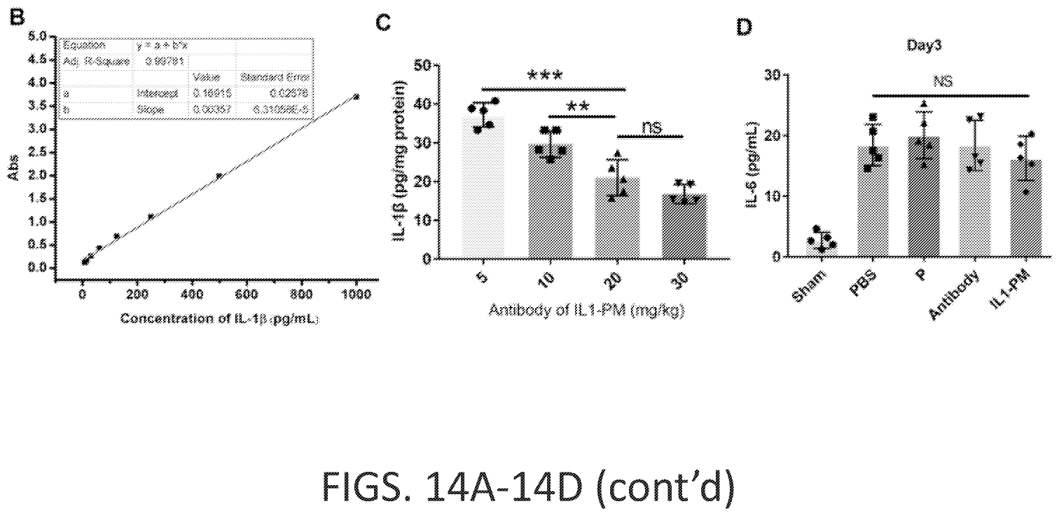

Infarct-homing ability of IL1-PMs. Next, the infarct-homing ability of IL1-PM was studied in C57BL/6 mice. The mouse MI model was first constructed through left anterior descending coronary (LAD) ligation, which has been widely used. The detection of IL1-PM migration was done in-vivo using a live imager. The mice were injected with either the Gevokizumab antibodies conjugated to the DSPE-PEG-NHS polymers or with IL1-PM. In both cases, the antibody ends were bound to Cy5.5 amine-reactive NHS-esters for signal detection. The IL1-PM@Cy5.5 and antibody@Cy5.5 were injected intravenously (i.v.) in the mice, with or without MI. Animals were imaged at indicated time intervals to determine the infarct-homing ability of the injected agents. As shown in FIG. 2A, 8 h after the injection of IL1-PM@Cy5.5, a sustained fluorescence signal was observed in the injured heart. The signal intensity grew from the 8 h to the 72 h time point. In contrast, no obvious fluorescence was observed in the non-MI heart, indicating the infarct signal driven migration of platelets to the heart area. In addition, the antibodies alone did not accumulate in the injured heart, further confirmed the infarct targeting ability of the platelet vehicle. The ex vivo tissue biodistribution was then imaged and analyzed (FIG. 2B). The infarcted hearts treated with IL1-PM@Cy5.5 showed stronger fluorescent signals than the IL1-PM@Cy5.5-treated non-MI hearts, the hearts treated with antibody@Cy5.5, and the other organs analyzed. In addition, most of non-platelet-conjugated antibodies (antibody@Cy5.5) accumulated in the kidneys by the study end point. The quantitative region-of-interest (ROI) analysis revealed that the IL1-PM@Cy5.5-treated infarcted hearts showed 8-fold higher fluorescence intensity than normal, non-infarcted hearts (FIG. 2C). In addition, the circulation lifetime of IL1-PMS and naïve platelets in normal mice was studied. As shown in FIG. 13, the concentrations of both platelets and IL1-PMs decreased with time. However, there is no significant difference between those two groups, indicating that DSPE-PEG-antibody modification has slight effects on circulation lifetime. Furthermore, all animals were subject to autopsy analysis upon euthanasia for signs of tumor growth in major organs and those tests have returned with no abnormal findings (FIG. 14A).

### Example 4

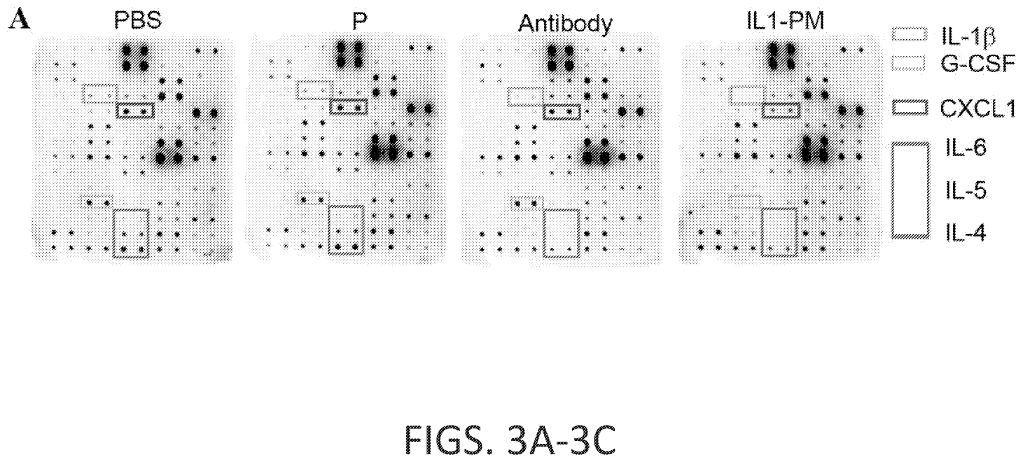

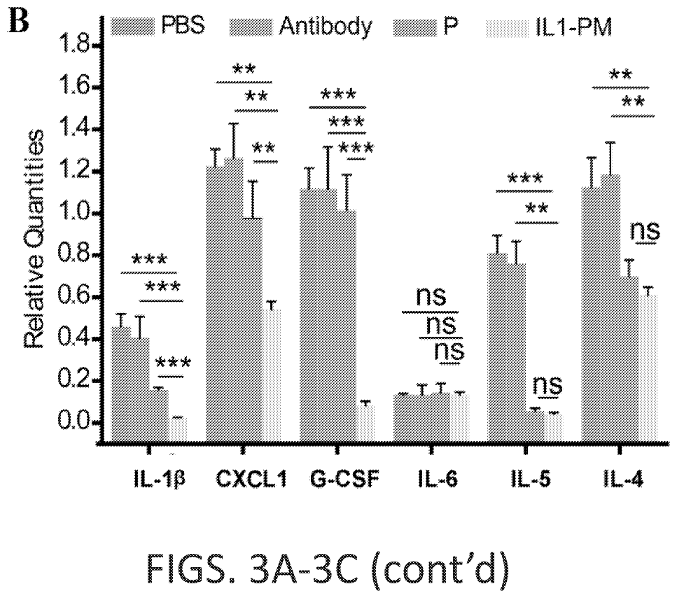

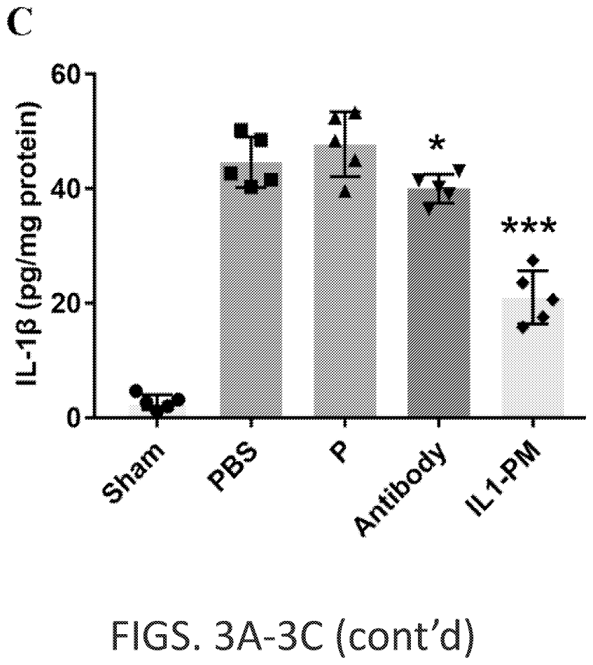

Anti-inflammatory outcomes of IL1-PM treatment. The in vivo anti-inflammatory ability of i.v. administered IL1-PM was next evaluated. To do this, the levels of inflammatory cytokines present in the blood and the hearts of mice were analyzed 3 days after treatment using a cytokine array. Four different treatment groups were compared: PBS, platelets (P), anti-IL-1β antibodies alone (Antibody), and IL1-PM. As shown in FIG. 3A, after correcting for background intensity and normalizing to the membrane's positive control, five cytokines/proteins were found significantly changed in mouse blood after Gevokizumab and IL1-PM treatment, including IL-1β, CXCL1, G-CSF, IL-5, and IL-4. Compared to the antibody group, the IL1-PM group significantly reduced the level of IL-1β, indicating the high affinity of the IL1-PM to the IL-1β (FIG. 3B).

Furthermore, the level of IL-1β were detected in treated heart tissues using ELISA. The results mirrored those of the blood detection results (FIG. 3C) and it seems that the neutralizing effects reached a plateau at 20 mg/kg and further increase in dose had no significant benefits (FIGS. 14A-14C). To that end, the dose of 20 mg/kg was used in the present study. Since IL-1β production leads to increased levels of IL-6, whether the neutralization of IL-1β reduced the levels of IL-6 was also assessed. As indicated by the cytokine array summarized in FIG. 3B, there was no significant difference in 11-6 expression levels among any of the treatment groups.

To further verify this, IL-6 expression was determined using an ELISA, which possesses a higher level of sensitivity than the cytokine array. The results were consistent with those of the cytokine array. Both the antibody and the IL1-PM treatments had a negligible effect on the IL-6 levels (FIG. 14D). This lack of dampening of IL-6 levels could be attributable to the complexity of the inflammatory response, in which many cytokines are involved, including IL-1α, IL-18 and TNF-α. Thus, embodiments of the present disclosure include targeting more than one cytokine in order to block the inflammatory cascade.

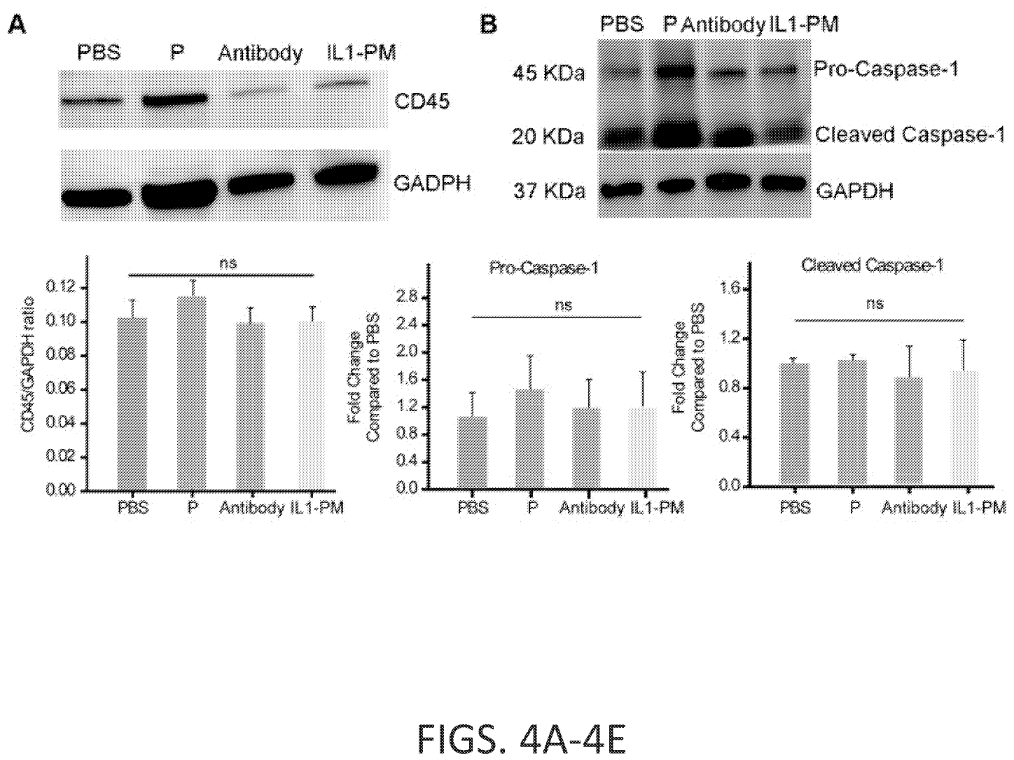

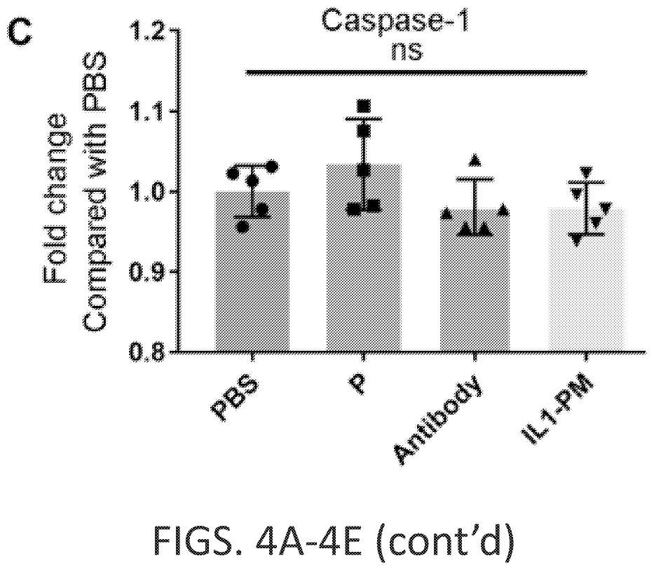

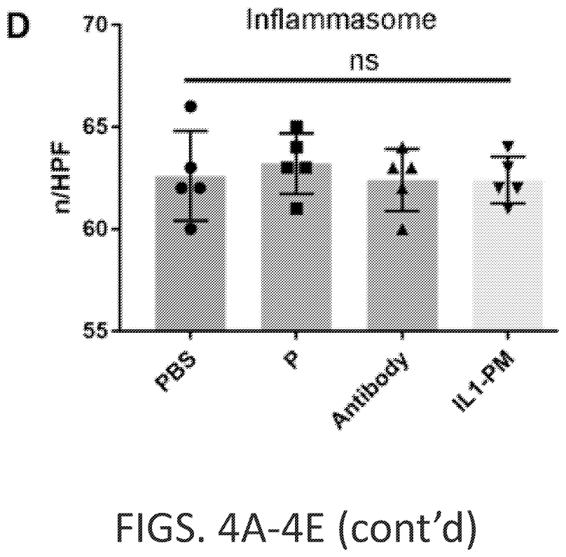

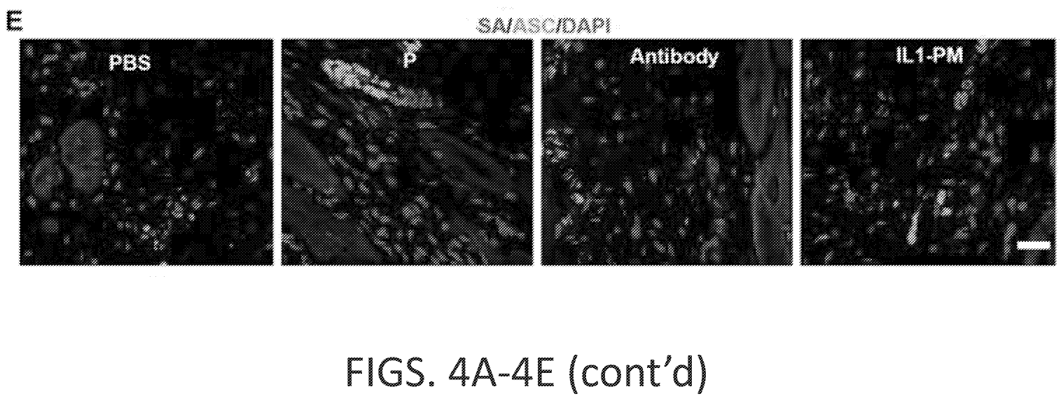

The anti-inflammatory effects of IL1-PM-mediated IL-1β neutralization were evaluated by quantifying the level of leukocyte infiltration in the injured heart. To do so, CD45, one of the most abundant leukocyte cell surface glycoproteins, was assessed. No obvious difference in the levels of CD45 expression was found in heart tissue among these four treatment groups (FIG. 4A). That may be because IL-1β may not be a key chemotaxis for leucocytes infiltrating during AMI in mice.

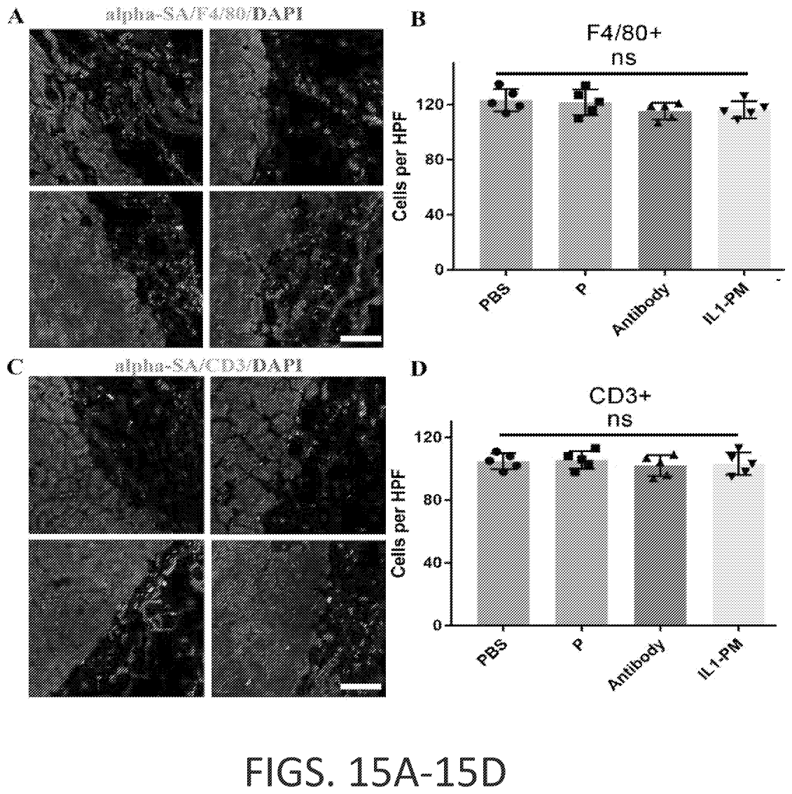

The next protein analyzed was caspase-1 since it is a key modulator of the inflammatory response to tissue injury, in addition to processing pro-IL-1β to its active, mature form, and inducing cardiac cell apoptosis. Caspase-1 activity was measured using two techniques: western blot and cleavage of a fluorogenic substrate. Both techniques indicated that neutralizing IL-1β had no effect on caspase-1 activity (FIGS. 4B-4C). Furthermore, the inhibition of inflammasome in the injured hearts as a result of the IL-1β neutralization was tested. Heart sections were immunoassayed for apoptosis-associated speck-like protein containing a caspase recruitment domain (ASC). However, no detectable differences were found in ASC expression between the four treatment groups, a result that was in accord with that of caspase-1 activity and CD45 expression (FIGS. 4D-4E). To further test the effects of IL-1β blockade on inflammatory response, the infiltration of macrophages and T cells in the infarct area was assessed. As shown in FIG. 15, neither macrophage (F4/80⁺) nor T cell (CD3⁺) infiltrations were inhibited after neutralizing IL-1β in mice via IL1-PM or treating them with just antibodies, which was consistent with the results for CD45 infiltration. Together, these results confirmed that IL-1β blockage had no significant effect on the formation of active inflammasomes or on the systemic inflammatory response. We found that neutralizing IL-1β did not change the level of caspase-1 activity as well, indicating that the attenuation of cardiac remodeling was independent of caspase-1 activity. The lack of correlation can be attribute to the fact that caspase-1 acts upstream of the IL-1β in the inflammatory pathway and it appears that neutralizing downstream IL-1β had no feedback effect on the caspase-1. These results prompted an investigation of the effects of IL-1β blockage on downstream enzymes instead.

### Example 5

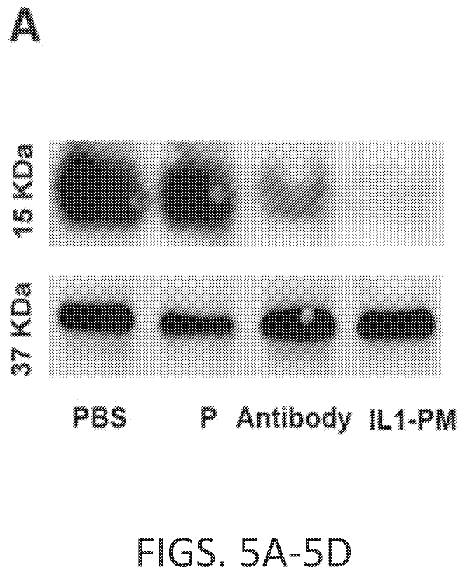

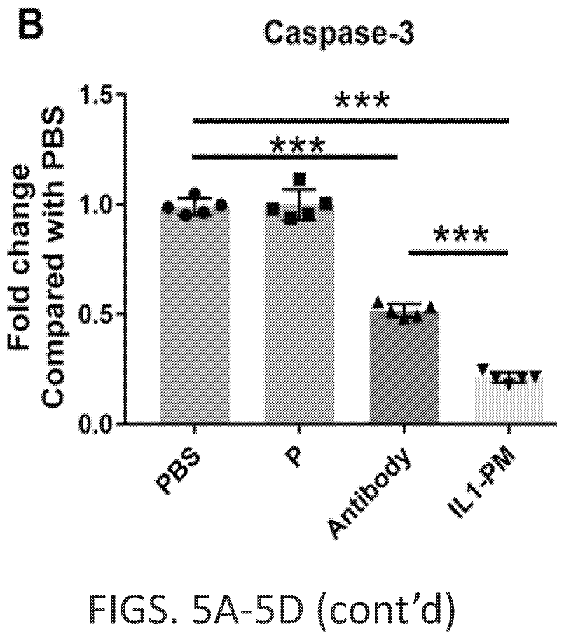

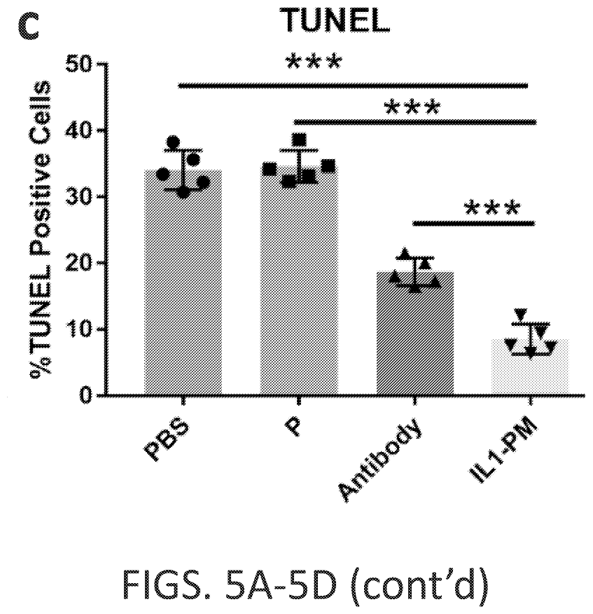

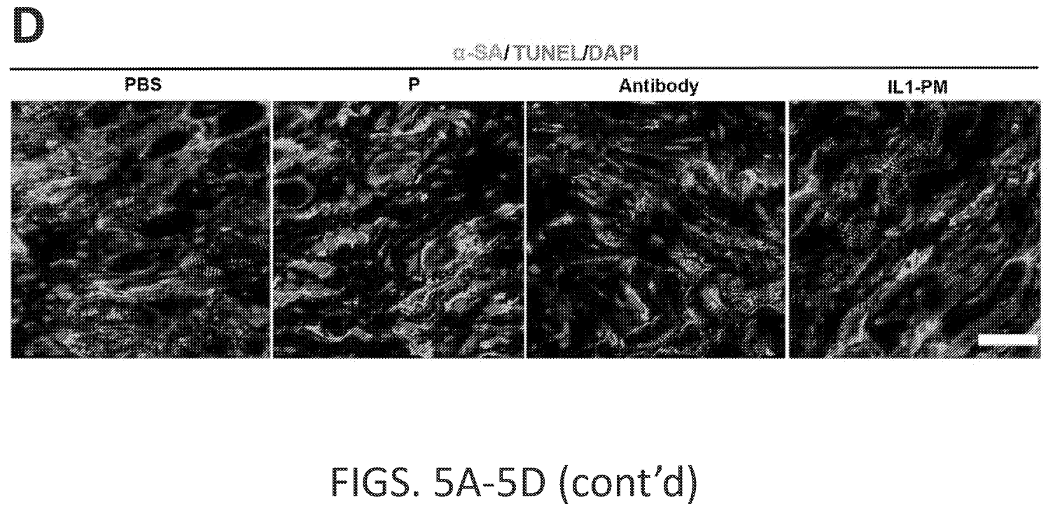

IL1-PMs inhibit cardiomyocyte apoptosis. Caspase-3 is an effector of apoptosis that is located downstream of IL-1β and is activated by it. To evaluate the detoxification efficiency of IL1-PM, caspase-3 activity was measured in the heart tissue, as a result of each of the four treatments, using western blot and caspase-3 fluorometric assays. FIG. 5A indicates that the level of cleaved caspase-3 (activated caspase-3) is reduced in the heart after antibody and IL1-PM treatment compared to the PBS and non-conjugated platelet controls. Furthermore, the IL1-PM treatment is a more effective inhibitor than the antibody treatment (FIG. 5B). In addition to the caspase-3, IL1-PM-driven inhibition of apoptosis was also detected using a TUNEL staining assay (FIGS. 5C-5D). As expected, the IL1-PM treatment had the highest impact on the apoptosis of cardiomyocytes when compared with the other treatment groups, including antibody treatment alone, a result that was consistent with the results of caspase-3 inhibition. From this data, it appears that the IL-1β-neutralizing ability of IL1-PM reduces caspase-3 activity, which then more efficiently inhibits cardiomyocyte apoptosis.

### Example 6

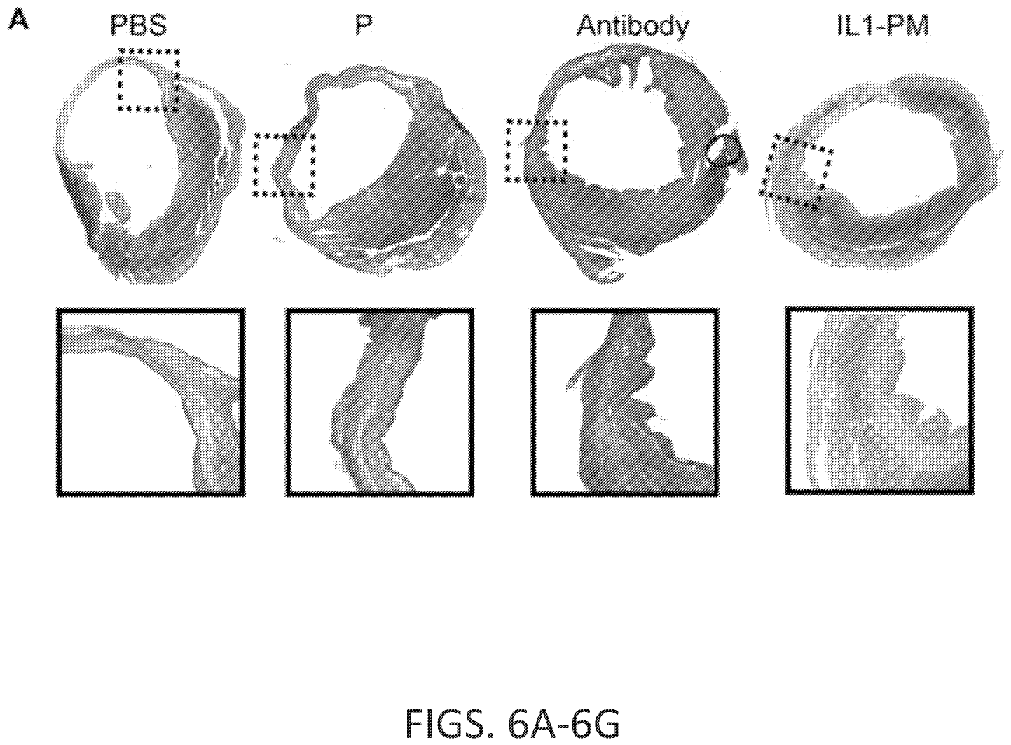

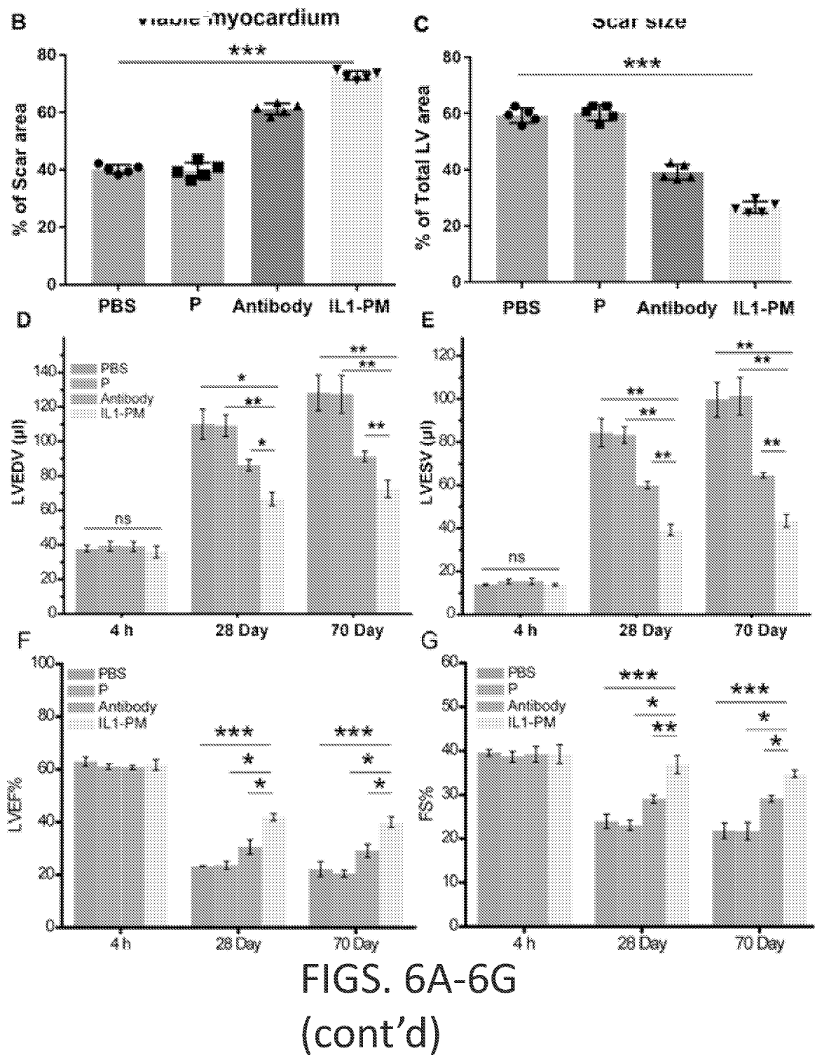

IL1-PMs attenuate of cardiac remodeling. After having demonstrated the capacity of IL1-PM to neutralize IL-1β and protect cardiomyocytes, its effects on cardiac remodeling were measured. First, changes in heart morphometry were investigated using Masson's trichrome staining, and reduced collagen accumulation in the scarred segment of the myocardium was observed (FIG. 6A). After quantification, it was determined that IL1-PM treatment was the most successful at protecting the heart and yielded more viable myocardium, with the smallest scar size, when compared to the antibody treated group or the controls (FIGS. 6B-6C). The attenuation of cardiac remodeling was reflected by a decrease in left ventricular end-diastolic volume (LVEDV), left ventricular end-systolic volume (LVESV), accompanied by an increased in left ventricular ejection fraction (LVEF) and fractional shortening (FS) as determined by transthoracic echocardiography (FIGS. 6D-6G). These results confirmed that the neutralization of IL-1β led to caspase-3 inhibition, reducing adverse cardiac remodeling after an AMI. Moreover, even though some benefits were noted from antibody therapy alone, the introduction of infarct-homing platelets improved the anti-inflammatory efficacy of the therapy.

What is claimed is:

1. A platelet-derived microparticle comprising a linker moiety and at least one therapeutic antibody.

2. The microparticle of claim 1, wherein the linker moiety is functionally coupled to the surface of the microparticle.

3. The microparticle of claim 1 or claim 2, wherein the linker moiety is functionally coupled to the surface of the microparticle via a lipophilic headgroup.

4. The microparticle of any of claims 1 to 3, wherein the linker moiety comprises 1,2-Distearoyl-sn-glycero-3-phosphorylethanolamine (DSPE) and a PEG polymer.

5. The microparticle of any of claims 1 to 4, wherein the PEG polymer is at least 5 kDa.

6. The microparticle of any of claims 1 to 5, wherein the linker moiety comprises an NHS-terminated DSPE-PEG polymer.

7. The microparticle of any of claims 1 to 6, wherein the at least one therapeutic antibody is covalently bound to the linker moiety.

8. The microparticle of any of claims 1 to 7, wherein the at least one therapeutic antibody neutralizes at least one aspect of an immune response.

9. The microparticle of any of claims 1 to 8, wherein the at least one therapeutic antibody is a monoclonal antibody.

10. The microparticle of any of claims 1 to 9, wherein the at least one therapeutic antibody binds to IL-1β.

11. The microparticle of claim 10, wherein the at least one antibody is Gevokizumab, Canakinumab, or derivatives, variants, or combinations thereof.

12. The microparticle of any of claims 1 to 11, wherein the microparticle comprises at least a second therapeutic antibody.

13. The microparticle of claim 12, wherein the second therapeutic antibody targets at least one of IL-1α, IL-16, IL-18, and TNF-α.

14. The microparticle of any of claims 1 to 13, wherein the microparticle is derived from inactivated platelets.

15. The microparticle of any of claims 1 to 14, wherein the microparticle further comprises at least one therapeutic agent.

16. A composition comprising a plurality of the microparticles of claim 1, and at least one pharmaceutically acceptable carrier or excipient.

17. The composition of claim 16, wherein the composition further comprises a physiologically suitable buffer.

18. The composition of claim 16 or 17, wherein the composition further comprises at least one therapeutic agent.

19. A method for treating a subject that has suffered a cardiac event, the method comprising administering the composition of any of claims 16 to 18.

20. The method of claim 19, wherein the composition is administered intravenously, subcutaneously, intracoronary, or intramuscularly or by surgical intervention.
